(19) United States
(12) Patent Application Publication
(10) Pub. No.: US 2017/0136152 A1
(43) Pub. Date: May 18, 2017

(54) GONAD-DERIVED SIDE POPULATION STEM CELLS

(71) Applicant: PRIMEGEN BIOTECH LLC, Irvine, CA (US)

(72) Inventors: Fariborz Izadyar, Santa Ana, CA (US); Jason Pachiarotti, Santa Ana, CA (US); Scott Greilach, Santa Ana, CA (US); Thomas C.K. Yuen, Santa Ana, CA (US)

(21) Appl. No.: 15/322,702

(22) PCT Filed: Jun. 30, 2015
(86) PCT No.: PCT/US2015/038630

§ 371 Date: Dec. 28, 2016

Related U.S. Application Data
(60) Provisional application No. 62/019,172, filed on Jun. 30, 2014.

(51) Int. Cl. A61L 27/38; C12N 5/0775; A61L 27/54; A61K 35/52; A61K 35/54; A61K 45/06; C12N 5/076; C12N 5/075

(52) U.S. Cl. CPC A61L 27/3834; C12N 5/061; C12N 5/0668; C12N 5/0609; A61K 35/52; A61K 35/54; A61K 45/06; A61L 27/3895; A61L 27/54; A61L 2430/34; A61L 2430/06; A61L 2430/02; A61L 2430/32; A61L 2430/20

(57) ABSTRACT

Compositions and methods for promoting tissue regeneration with gonad-derived stem cell side population cells are provided.

### CROSS REFERENCE TO RELATED APPLICATION

This application claims the benefit of U.S. Provisional Patent Application No. 62/019,172, filed Jun. 30, 2014, the entire disclosure of which is herein incorporated by reference.

### FIELD

Disclosed herein are compositions including substantially homogenous populations of gonad-derived stem cell side population cells, for promoting tissue regeneration and therapy of disease.

### BACKGROUND

Stem cells have been shown to repopulate and repair tissues, organs and/or organ systems. Of interest for regenerative medicine is the use of adult or post-natal stem cell side-population cells for cell-based therapies.

### SUMMARY

Disclosed herein are compositions comprising substantially homogenous populations of gonadal-derived stem cell side population cells for use in tissue regeneration.

In certain embodiments, compositions are provided comprising substantially homogenous populations of isolated gonadal-derived stem cell side population cells (GDSC-SP) and at least one pharmaceutically acceptable excipient. In other embodiments, at least about 85% of the cells in the substantially homogenous population of GDSC-SP cells express all of the cell surface markers SSEA4, ABCG2, CD117, CD34, BCRP1, SCA1, CD90, CD49f, VASA, GPR-125 and do not express CD45 or lineage markers. In other embodiments, the composition further comprises at least one bioactive agent such as a growth factor, an anti-rejection agent, an anti-inflammatory agent, an anti-infective agent (e.g., antibiotics and antiviral agents), an analgesic and/or analgesic combination, an anti-asthmatic agent, an anticonvulsant, an antidepressant, an anti-diabetic agent, an anti-neoplastic, an anti-cancer agent, an anti-psychotic, an antioxidant, an immunosuppressive agent, a vitamin, a mineral, or an agent used for cardiovascular diseases such as an anti-restenosis and/or anti-coagulant compound. In yet other embodiments, the bioactive agent is an immunosuppressive agent.

Also disclosed herein is a method of promoting tissue regeneration in a subject in need thereof comprising administering a tissue regenerating effective amount of a substantially homogenous population of GDSC-SP cells to a treatment site in the subject thereby inducing tissue regeneration at the treatment site, wherein at least about 85% of the cells in the substantially homogenous population of GDSC-SP cells express all of the cell surface markers SSEA4, ABCG2, CD117, CD34, BCRP1, SCA1, CD90, CD49f, VASA, GPR-125, and do not express CD45 or lineage markers. In other embodiments, the composition comprises a substantially homogenous population of GDSC-SP and a pharmaceutically acceptable carrier. In other embodiment, the a substantially homogenous population of GDSC-SP are either autologous or allogeneic to the subject.

In other embodiments, the tissue regeneration is skin regeneration at the site of a wound, cardiac muscle regeneration, cartilage and tendon regeneration, bone regeneration, neural tissue regeneration, blood and vascular regeneration, or the tissue regeneration minimizes scarring at a wound site.

In certain embodiments, the tissue regenerating effective amount of the substantially homogenous population of GDSC-SP is approximately 0.5×10⁶ cells/10 mm of treatment site per treatment location per day. In other embodiments, the administering step comprises at least one method selected from the group consisting of topical application, intradermal injection, intravenous injection, and subcutaneous injection.

In yet other embodiments, the substantially homogenous population of GDSC-SP are expanded in culture in a clinical grade culture media which includes growth promoting factors and supplements including, but not limited to, FGF, GDNF, EGF, LIF, IGF, PDGF, EPO, GM-CSF, platelet rich plasma (PRP), and human serum to form a substantially homogenous GDSC-SP cell line.

In other embodiments, the method further comprises differentiating the substantially homogenous population of GDSC-SP into cells of the same tissue type as the tissue in need of regeneration.

In other embodiments, the composition further comprises at least one bioactive agent such as a growth factor, an anti-rejection agent, an anti-inflammatory agent, an anti-infective agent (e.g., antibiotics and antiviral agents), an analgesic and/or analgesic combination, an anti-asthmatic agent, an anticonvulsant, an antidepressant, an anti-diabetic agent, an anti-neoplastic, an anti-cancer agent, an anti-psychotic, an antioxidant, an immunosuppressive agent, a vitamin, a mineral, or an agent used for cardiovascular diseases such as an anti-restenosis and/or anti-coagulant compound. In some embodiments, the bioactive agent comprises a growth factor. In other embodiments, the bioactive agent comprises an immunosuppressive agent. In yet other embodiments, the bioactive agent is administered by a route comprising systemic administration or local administration at the site of tissue regeneration.

### BRIEF DESCRIPTION OF THE DRAWINGS

FIG. 1 depicts the side population (SP) in human male germline stem cells. Cultured human male germ line stem cells after several passages contain 40% SSEA4 and small population of SP cells as detected by ABCG2 antibody. FIG. 1A—Unstained controls; FIG. 1B—isotype control; FIG. 1C—Alexa-488-anti-ABCG2 and PE-anti-SSEA.

FIG. 2 depicts the SP in female germline stem cells. Cultured murine female germline stem cells after several passages contained 50% SP cells as detected by Hoechst staining (FIG. 2B) with verapamil controls (FIG. 2A).

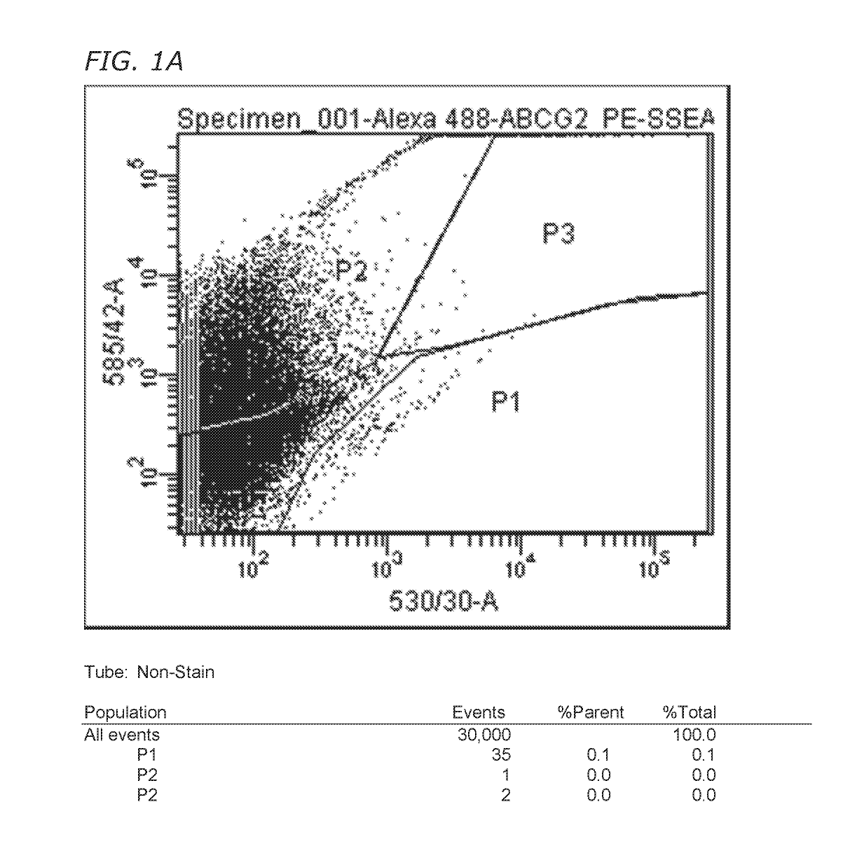

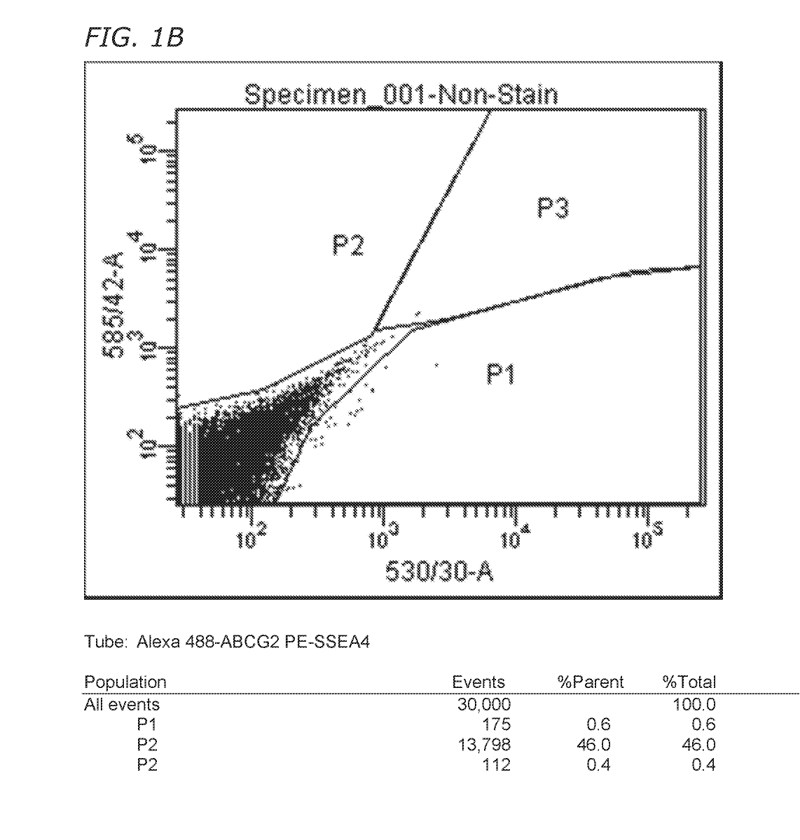

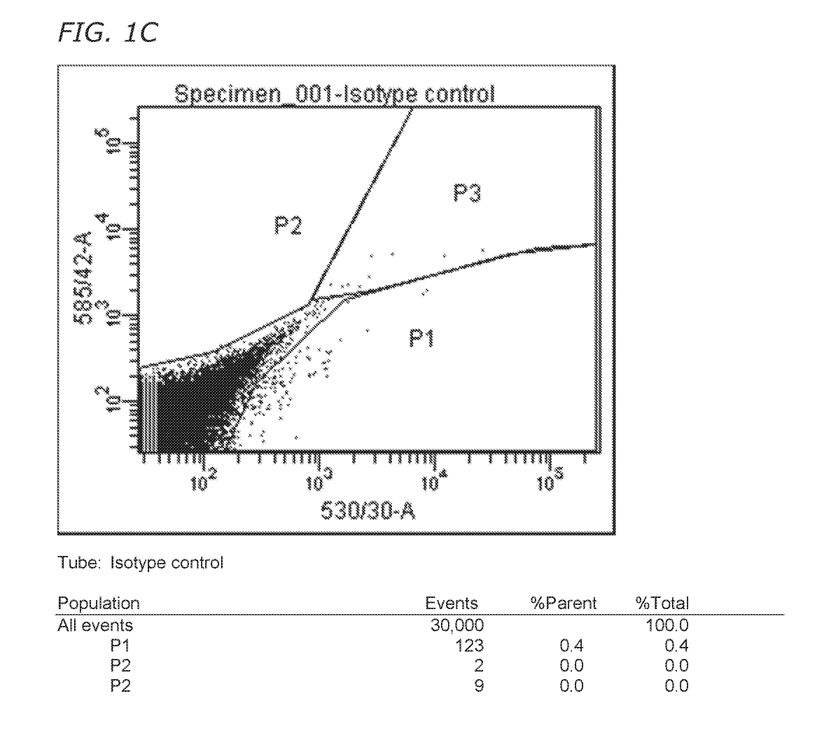

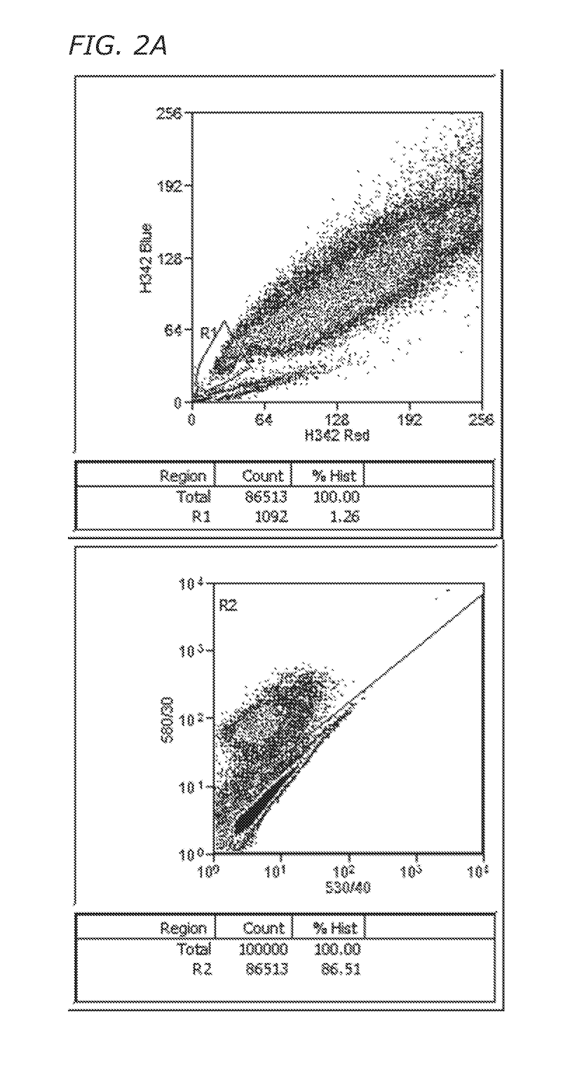

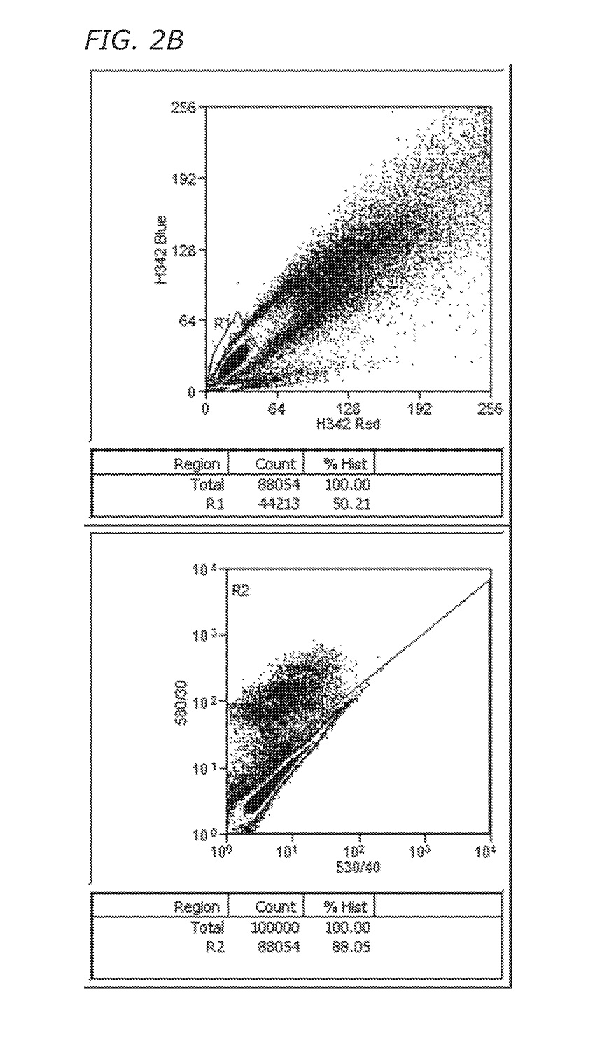

### DETAILED DESCRIPTION

The present disclosure provides substantially homogenous populations of gonad-derived stem cell side population (GDSC-SP) cells from individuals for tissue regeneration and treatment of disease.

The term “mammal” as used herein, encompasses any mammal, such as a mammal in need of such treatment or prevention. Examples of mammals include, but are not limited to, cows, horses, sheep, pigs, cats, dogs, mice, rats, rabbits, guinea pigs, monkeys, etc., more preferably, a human.

With the moral and ethical considerations of developing cell lines from human embryonic cells, the search for alternative sources of stem cells is underway. The alternative sources of adult stem cells have been found in many tissue types, including umbilical cord blood; mesenchymal tissue; skin; brain; bone marrow; adipose tissue amniotic tissue and gonads.

Most stem cell preparations from whole tissue are a mixture of cells consisting of the stem cells and non-stem cells. More often than not, the non-stem cell population is much more abundant. Procedures to isolate stem cells are becoming much more prevalent and provide purified fractions of stem cells. Most isolation procedures include use of antibodies, nuclear dyes, or magnetic beads. However, a subpopulation of stem cells, stem cell side population cells, is of particular interest.

Different subpopulations of germline stem cells have been identified and characterized in mammalian gonads (see co-pending US 2010/0285577). Described herein is a different gonad-derived stem cell population, the gonad-derived stem cell side population.

The gonad-derived stem cell side population (GDSC-SP) is a cell population found within gonadal tissue that is multipotent and suitable for use in tissue regeneration applications.

In flow cytometry, a side population (SP) is a sub-population of cells that is distinct from the main population on the basis of the markers employed. By definition, cells in a side population have distinguishing biological characteristics (for example, they may exhibit stem cell-like characteristics), but the exact nature of this distinction depends on the markers used in identifying the side population. Side populations have been identified in cancer and may be the cells that efflux chemotherapy drugs, accounting for the resistance of cancer to chemotherapy. In testicular stem cells more than 40% of the SP (defined as cells that show higher efflux of DNA-binding dye Hoechst 33342) were undifferentiated spermatogonia, while other differentiated fractions were represented by only 0.2%. The molecules involved in effluxing Hoechst 33342 are members of the ATP-binding cassette (ABC) family, such as MDR1 (P-glycoprotein) and ABCG2.

Side population cells can be identified based on the passive uptake of Hoechst 33342 DNA dye by live cells of interest and efflux of the Hoechst 33342. Stem cells and early progenitors are able to pump out Hoechst via the ABC transporters allowing the observation of a cell population that has a Hoechst low fluorescence in both blue and red regions of the spectrum. Propidium Iodide (PI) is added to exclude dead cells and its bright red fluorescence is collected with the same detector as Hoechst red fluorescence. ABC pumps can be specifically inhibited by drugs such as verapamil or reserpine and samples treated with one of these drugs before incubation with Hoechst can show loss of SP phenotype and can be used as control to confirm SP identification. SP phenotype depends on Hoechst concentration, incubation time, and temperature stability. These conditions can vary with cell type.

The term “gonad-derived stem cell” refers to a population of gonadal cells found in post-natal mammals that are multipotent and have the potential to differentiate into a variety of cell types. Gonadal SP, gonad-derived SP and GDSC-SP cells all refer to the same subpopulation of GDSC that have the phenotype of positive for all of the markers ABCG2 (ATP-binding cassette subfamily G member 2; CDw338; BRCP1), SCA1, CD90, CD49f, SSEA4, VASA, and CPR125, and negative for CD45 and lineage markers. The GDSC-SP also have low expression of CD117 and CD34. Lineage is defined for murine cells as the markers CD3e, CD11c, CD45R/B220, Ly-76, Ly-6G and Ly-6C and for human cells, the markers CD2, CD3, CD14, CD16, CD19, CD24, CD56, CD66b and glycophorin A. Lineage negative cells do not express any of the lineage markers. Gonadal cells can be obtained from both male and female sources. In one embodiment, the GDSC-SP cells are testes-derived stem cell side population cells. In another embodiment, the GDSC-SP ovary-derived stem cell side population cells.

As used herein, the term “substantially homogeneous” refers to a population of isolated GDSC-SP which are purified such that at least 85% of the cells in the population express all of the markers ABCG2 (ATP-binding cassette subfamily G member 2; CDw338; BRCP1), SCA1, CD90, CD49f, SSEA4, VASA, and CPR125, and are negative for CD45 and for lineage markers. In other embodiments, at least about 90%, at least about 92%, at least about 95%, or at least 97% of the cells in a substantially homogeneous population of isolated GDSC-SP have the desired phenotype,

The GDSC-SP cells are particularly suitable for use in tissue regeneration. Additionally, gonadal SP cells can differentiate into osteogenic, chondrogenic, cardiogenic, neurogenic, and adipogenic cells. Bulk isolation of gonad-derived stem cells can differentiate into multiple mesenchymal lineages. Gonad-derived SP cells may be gonadal precursor cells with a high degree of plasticity and lay quiescent until some signal stimulates them to commit to a specific lineage in response to apoptosis, cellular damage, or for tissue homeostasis.

Gonad-derived SP cells, isolated from mouse testes tissue and sorted into culture, remain undifferentiated, are in a quiescent state, retain a high level of plasticity in vitro and have the ability to differentiate into different cell types in vitro.

In other embodiments, quiescent state GDSC-SP cells can be stimulated from a quiescent state by stimulation with a low-level laser resulting in the proliferation of the CDSC-SP. An exemplary low-level laser is an A1 GaAs laser. For example, the laser may be a pulsed laser, such as the pulsed tunable laser (e.g., a Kerr-lens self-mode-locked (KLM) titanium sapphire (Ti:S) laser) described in US 20100/265493, incorporated herein by reference for all it discloses regarding pulsed tunable lasers. In another embodiment, the laser may be a calibrated laser. Methods and equations for calibrating various parameters of a pulsed laser (e.g., pulse rate, pulse energy, etc.) may be similar to those described for use in eye surgery, adapted for use in treating organs as described herein. Such methods are described, for example, in US 2011/0267446, US 2007/0213697, US 2011/0028956, all of which are incorporated herein by reference for all they disclose regarding calibrated lasers. Low-level lasers are further described in, for example, US 2003/0144712, US 2003/0212442, US 2004/0220513, US 2004/0260367, US 2004/0153130, and US 2010/0016783, all of which are incorporated herein by reference for all they disclose regarding low-level lasers. Additional description of lasers, including continuous wave lasers, useful in the methods described herein, may be found, for example, in US 2012/0302879, US 2013/0211388, US 2013/0184693, and US 2013/0102880, all of which are incorporated herein by reference for all they disclose regarding lasers, including continuous wave lasers.

In a further embodiment, a laser useful in the methods disclosed herein comprises a wavelength in the infrared (IR) or near-infrared (NIR) spectrum, for example, having a wavelength of about 600-1100 nm, including ranges of about 600-700 nm, 650-750 nm, 700-800 nm, 750-850 nm, 800-900 nm, 850-950 nm, 900-1000 nm, 950-1050 nm, 1000-1100 nm, 650-1050 nm, 700-1000 nm, or 750-950 nm, or including about 600 nm, 620 nm, 625 nm, 650 nm, 675 nm, 700 nm, 725 nm, 750 nm, 775 nm, 780 nm, 785 nm, 790 nm, 800 nm, 810 nm, 820 nm, 825 nm, 850 nm, 875 nm, 900 nm, 925 nm, 950 nm, 975 nm, 1000 nm, 1025 nm, 1050 nm, 1075 nm, or 1100 nm.

In other embodiments, the laser comprises a power output of about 1-500 mW, including ranges of about 1-5 mW, 1-10 mW, 1-15 mW, 1-20 mW, 1-25 mW, 1-30 mW, 1-35 mW, 1-40 mW, 1-45 mW, 1-50 mW, 1-75 mW, 1-100 mW, 5-10 mW, 10-15 mW, 15-20 mW, 20-25 mW, 25-30 mW, 30-35 mW, 35-40 mW, 40-45 mW, 45-50 mW, 5-45 mW, 10-40 mW, 15-35 mW, 20-30 mW, 50-150 mW, 100-200 mW, 150-250 mW, 200-300 mW, 250-350 mW, 300-400 mW, 350-450 mW, 400-500 mW, 50-450 mW, 100-300 mW, or including about 3 mW, about 5 mW, about 10 mW, about 15 mW, about 20 mW, about 25 mW, about 30 mW, about 35 mW, about 40 mW, about 45 mW, about 50 mW, about 60 mW, about 70 mW, about 80 mW, about 90 mW, about 100 mW, about 150 mW, about 200 mW, about 250 mW, about 300 mW, about 350 mW, about 400 mW, about 450 mW, or about 500 mW.

In other embodiments, the laser emits a beam area of about 4 mm² or about 0.1256 cm². In another embodiment, the laser may emit a beam area of about 0.1-10 mm², including ranges of about 0.5-9.5 mm², 1-9 mm², 2-8 mm², 3-7 mm², 4-6 mm², 1-7 mm², 2-6 mm², 3-5 mm², 0.1-1 mm², 1-2 mm², 2-3 mm², 3-4 mm², 4-5 mm², 5-6 mm², 6-7 mm², 8-9 mm², 9-10 mm², or about 0.1 mm², 0.5 mm², 1 mm², 1.5 mm², 2 mm², 2.5 mm², 3 mm², 3.5 mm², 4 mm², 4.5 mm², 5 mm², 5.5 mm², 6 mm², 6.5 mm², 7 mm², 7.5 mm², 8 mm², 8.5 mm², 9 mm², 10 mm² or more.

In other embodiments, the energy is applied for about 1-120 seconds, including ranges of about 1-5, 1-10, 1-20, 1-25, 1-30, 1-35, 1-40, 1-45, 1-50, 1-60, 1-70, 1-80, 1-90, 1-100, 1-110, 5-10, 10-15, 15-20, 20-25, 25-30, 35-40, 45-50, 50-55, 55-60, 60-70, 70-80, 80-90, 90-100, 100-110, or 110-120 seconds, or about 1, 2, 3, 4, 5, 10, 15, 20, 25, 30, 35, 40, 45, 50, 55, 60, 65, 70, 75, 80, 85, 90, 95, 100, 105, 110, 115, or 120 seconds.

In other embodiments, the laser has a power density (irradiance) of between about 1 mW-5 W/cm², including ranges of about 1-5, 1-10, 1-15, 1-20, 1-25, 1-30, 1-35, 1-40, 1-45, 1-50, 1-75, 1-100, 1-150, 1-200, 1-250, 1-300, 1-350, 1-400, 1-450, 1-500, 1-600, 1-700, 1-800, 1-900, 1-1000, 1-2000, 1-3000, 1-4000, 5-10, 10-20, 20-30, 30-40, 40-50, 50-100, 100-200, 200-300, 300-400, 400-500, 500-1000, 1000-2000, 2000-3000, 3000-4000, or 4000-5000, or about 1, 5, 10, 15, 20, 25, 30, 35, 40, 45, 50, 75, 100, 150, 200, 250, 300, 350, 400, 450, 500, 600, 700, 800, 900, 1000, 2000, 3000, 4000, or 5000 mW/cm².

In some embodiments, GDSC-SP cells are isolated from gonadal tissue by a method comprising obtaining gonadal tissue tissue from a mammal, forming a cell suspension of gonadal cells, staining the gonadal cells with Hoechst 33342 dye and isolating a side population of cells from the Hoechst-stained gonadal cells. In other embodiments, GDSC-SP can be isolated by magnetic beads conjugated with cell surface markers including but not limited to SSEA4 and ABCG2.

In other embodiments, the GDSC-SP cells are less than 4 microns in size, in other embodiments, the GDSC-SP cells are less than 3 microns in size, or less than 2 microns in size. In still further embodiments, the GDSC-SP are from about 0.5-4 microns in size, from about 0.5-3 microns in size, from about 0.5-2 microns in size, from about 1-3 microns in size, or from about 1-2 microns in size. In certain embodiments, the GDSC-SP cells are isolated based on both Hoechst efflux and size.

Furthermore, GDSC-SP cells may exhibit immune privilege when introduced into an allogeneic subject. GDSC-SP do not activate T cells and will not stimulate T cell proliferation and thus do not trigger the immune system. Therefore it may be possible to establish banks, cell lines, or other renewable sources of GDSC-SP to allow their use as “off the shelf” compositions for the treatment of disease. In another embodiment, GDSC-SP may exert immune suppressive and immune modulatory effects. Co-infusion of GDSC-SP cells during cell or organ transplantation may reduce the chance of immune rejection and graft-versus-host disease (GVHD) by inhibiting the host immune response against the transplanted cells.

In other embodiments, GDSC-SP exert angiogenic and anti inflammatory effects, such that injection of GDSC-SP cells results in reduced inflammation, new vascularization, and improvement of blood supply at an injection site.

The present disclosure also provides GDSC-SP cells for tissue regeneration. In an embodiment, the GDSC-SP cells are autologous to the recipient subject. In other embodiments, the GDSC-SP are allogeneic to the recipient subject.

Thus, in certain embodiments, methods are provided for using allogeneic substantially homogeneous populations of GDSC-SP cells in the treatment of disease, such as by tissue regeneration.

In one embodiment, the substantially homogeneous population of GDSC-SP cells are used in skin tissue regeneration and/or wound healing. In a non-limiting example, approximately 0.5×10⁶ to approximately 5.0×10⁶ GDSC-SP cells/10 mm of treatment site per treatment location per day are injected adjacent to, or within the desired treatment location or site. The GDSC-SP cells aid in tissue regeneration by increasing vascularization, increasing cell migration to the site of injury, decreasing the amount of scarring and increasing tissue regeneration. GDSC-SP cells also decrease the healing time of wounds, thereby decreasing the possibility of infection. In addition, certain embodiments disclosed herein aid in wound healing in patients with chronic diseases such as diabetes.

The substantially homogeneous population of GDSC-SP cells provides tissue regeneration for the treatment of both acute and chronic wounds. Acute wounds are those wounds that heal promptly, within 30 days (or 60 days in diabetics). Non-limiting examples of acute wounds that can be treated with the present invention include abrasions, avulsions, contusions, crush wounds, cuts, lacerations, projectile wounds and puncture wounds. Chronic wounds include, but are not limited to, diabetic skin sores, pressure sores, surgical wounds, spinal injury wounds, burns, chemical-induced wounds and wounds due to blood vessel disorders.

An advantage of the present substantially homogeneous population of GDSC-SP cells and compositions is that they promote tissue healing by regeneration of like tissues rather than scar formation.

In an embodiment, a method is provided in which the substantially homogeneous population of GDSC-SP cells is differentiated prior to use in tissue regeneration. In a non-limiting example, for tissue regeneration of cardiac tissue after myocardial infarction, it is possible to differentiate the GDSC-SP into a cardiogenic precursor cell or cardiomyocyte prior to transplantation of the cells to the treatment site. In another embodiment, GDSC-SP cells can be differentiated into neurons and be used for neurodegenerative diseases including Parkinson's disease and Alzheimer's disease, repair of sensory neurons and auditory neurons.

In yet another embodiment, the substantially homogeneous population of GDSC-SP cells can be used for chondrocyte differentiation, osteocyte differentiation, cartilage repair, bone repair and osteoarthritis.

The present disclosure also encompasses compositions comprising the substantially homogeneous population of GDSC-SP cells in a suitable carrier. In one embodiment, the composition also comprises a bioactive agent. In other embodiments, the substantially homogeneous population of GDSC-SP cell-containing composition is co-administered with one or more bioactive agents. By “co-administration” is meant administration before, concurrently with, e.g., in combination with bioactive agents in the same formulation or in separate formulations, or after administration of a therapeutic composition as described above.

As used herein, the phrase, “bioactive agents” refers to any organic, inorganic, or living agent that is biologically active or relevant. For example, a bioactive agent can be a protein, a polypeptide, a polysaccharide (e.g. heparin), an oligosaccharide, a mono- or disaccharide, an organic compound, an organometallic compound, or an inorganic compound. It can include a living or senescent cell, bacterium, virus, or part thereof. It can include a biologically active molecule such as a hormone, a growth factor, a growth factor producing virus, a growth factor inhibitor, a growth factor receptor, an anti-inflammatory agent, an antimetabolite, an integrin blocker, or a complete or partial functional insense or antisense gene. It can also include a man-made particle or material, which carries a biologically relevant or active material. An example is a nanoparticle comprising a core with a drug and a coating on the core.

Bioactive agents also can include drugs such as chemical or biological compounds that can have a therapeutic effect on a biological organism. Non-limiting examples include, but are not limited to, growth factors, anti-rejection agents, anti-inflammatory agents, anti-infective agents (e.g., antibiotics and antiviral agents), analgesics and analgesic combinations, anti-asthmatic agents, anticonvulsants, antidepressants, anti-diabetic agents, anti-neoplastics, anticancer agents, anti-psychotics, antioxidants, immunosuppressive agents, vitamins and minerals, and agents used for cardiovascular diseases such as anti-restenosis and anti-coagulant compounds.

Bioactive agents also can include precursor materials that exhibit the relevant biological activity after being metabolized, broken-down (e.g. cleaving molecular components), or otherwise processed and modified within the body. These can include such precursor materials that might otherwise be considered relatively biologically inert or otherwise not effective for a particular result related to the medical condition to be treated prior to such modification.

Combinations, blends, or other preparations of any of the foregoing examples can be made and still be considered bioactive agents within the intended meaning herein. Aspects of the present disclosure directed toward bioactive agents can include any or all of the foregoing examples.

In one embodiment, the bioactive agent is a growth factor. A growth factor is any agent which promotes the proliferation, differentiation and functionality of the implanted GDSC-SP cells. Non-limiting examples of suitable growth factors include leukemia inhibitory factor (LIF), epidermal growth factor (EGF), fibroblast growth factor (FGF), transforming growth factor-beta (TGF-β), insulin-like growth factor (IGF), and vascular endothelial growth factor (VEGF), human growth hormone, platelet-derived growth factor (PDGF) interleukins, cytokines, and combinations thereof.

In one embodiment, the bioactive agent is an immunosuppressive agent. An immunosuppressive agent is an agent is any agent that prevents, delays the occurrence of, or decreases the intensity of, the desired immune response, e.g., rejection of a transplanted cell, tissue, or organ. Certain immunosuppressive agents which suppress cell-mediated immune responses against cells identified by the immune system as non-self. Examples of immunosuppressive agents include, but are not limited to, cyclosporin, cyclophosphamide, prednisone, dexamethasone, methotrexate, azathioprine, mycophenolate, thalidomide, FK-506, systemic steroids, as well as a broad range of antibodies, receptor agonists, receptor antagonists, and other such agents as known to one skilled in the art.

As used herein, “immunosuppression” refers to prevention of the immune response (for example by the administration of an “immunosuppresive agent”, as defined herein) such that an “immune response”, as defined herein, is not detectable. As used herein, “prevention” of an immune response means an immune response is not detectable. An immune response (for example, transplant rejection or antibody production) is detected according to methods well-known in the art and defined herein.

“Immunosuppression” also means a delay in the occurrence of the immune response as compared to any one of a transplant recipient that has not received an immunosuppresive agent, or a transplant recipient that has been transplanted with material that is not “immunologically blinded” or “immunoprivileged”, as defined herein. A delay in the occurrence of an immune response can be a short delay, for example 1 hr-10 days, i.e., 1 hr, 2, 5 or 10 days. A delay in the occurrence of an immune response can also be a long delay, for example, 10 days-10 years (i.e., 30 days, 60 days, 90 days, 180 days, 1, 2, 5 or 10 years).

“Immunosuppression” also means a decrease in the intensity of an immune response. The intensity of an immune response can be decreased such that it is 5-100%, preferably, 25-100% and most preferably 75-100% less than the intensity of the immune response of any one of a transplant recipient that has not received an immunosuppresive agent, or a transplant recipient that has been transplanted with material that is not autologous. The intensity of an immune response can be measured by determining the time point at which transplanted material is rejected. For example, an immune response comprising rejection of transplanted material at day 1, post-transplantation, is of a greater intensity than an immune response comprising the rejection of transplanted material at day 30, post-transplantation. The intensity of an immune response can also be measured by quantitating the amount of a particular antibody capable of binding to the transplanted material, wherein the level of antibody production correlates directly with the intensity of the immune response. Alternatively, the intensity of an immune response can be measured by determining the time point at which a particular antibody capable of binding to the transplanted material is detected.

Various strategies and agents can be utilized for immunosuppression. For example, the proliferation and activity of lymphocytes can be inhibited generally with agents such as, for example, FK-506, or cyclosporin or other immunosuppressive agents. Another possible strategy is to administer an antibody, such as an anti-GAD65 monoclonal antibody, or another compound which masks a surface antigen on a transplanted cell and therefore renders the cell practically invisible to the immune system of the host.

In yet another embodiment, the substantially homogeneous population of GDSC-SP cells are administered with hyperbaric oxygen therapy for the treatment of chronic wounds.

In an embodiment, the substantially homogeneous population of GDSC-SP cells are administered with skin grafts to aid in the grafting process and with tissue regeneration.

The substantially homogeneous population of GDSC-SP cells, or cells differentiated therefrom, may be transplanted into the recipient where the cells will proliferate and differentiate to form new cells and tissues thereby providing the physiological processes normally provided by that tissue. The term “transplanted” as used herein refers to transferring cells alone or cells that are embedded in a support matrix. As used herein, the term “tissue” refers to an aggregation of similarly specialized cells united in the performance of a particular function. Tissue is intended to encompass all types of biological tissue including both hard and soft tissue. Soft tissue refers to tissues that connect, support, or surround other structures and organs of the body. Soft tissue includes muscles, tendons (bands of fiber that connect muscles to bones), fibrous tissues, fat, blood vessels, nerves, and synovial tissues (tissues around joints). Hard tissue includes connective tissue (e.g., hard forms such as osseous tissue or bone) as well as other muscular or skeletal tissue.

In another embodiment, the substantially homogeneous population of GDSC-SP cells are administered with a pharmaceutically acceptable carrier or excipients. The pharmaceutically acceptable excipients described herein, for example, vehicles, adjuvants, carriers or diluents, are well-known to those who are skilled in the art and are readily available to the public. It is preferred that the pharmaceutically acceptable carrier or excipient be one which is chemically inert to the therapeutic composition and one which has no detrimental side effects or toxicity under the conditions of use.

The choice of excipient or carrier will be determined in part by the particular therapeutic composition, as well as by the particular method used to administer the composition. Accordingly, there are a wide variety of suitable formulations of the pharmaceutical compositions. The formulations described herein are merely exemplary and are in no way limiting.

Often the physiologically acceptable carrier is an aqueous pH buffered solution. Examples of physiologically acceptable carriers include, but are not limited to, saline, solvents, dispersion media, cell culture media, aqueous buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The present disclosure further provides compositions useful in practicing the therapeutic methods. A subject composition includes, in admixture, a pharmaceutically acceptable excipient (carrier) or media and the substantially homogeneous population of GDSC-SP cells, including tissues derived therefrom, alone or in combination with one or more bioactive agents, and at a strength effective for administration by various means to a patient experiencing cellular or tissue loss or deficiency.

It is a still further embodiment to provide compositions for use in methods which comprise, or are based upon, the substantially homogeneous population of GDSC-SP cells, including lineage-uncommitted populations of cells, lineage-committed populations of cells or tissues derived therefrom, along with a pharmaceutically acceptable carrier or media. Also contemplated are compositions comprising bioactive agents that act on or modulate the GDSC-SP cells and/or tissues derived therefrom, along with a pharmaceutically acceptable carrier or media.

The preparation of cellular or tissue-based compositions is well understood in the art. Such compositions may be formulated in a pharmaceutically acceptable media. The cells may be in solution or embedded in a matrix. The preparation of compositions with bioactive agents (such as, for example, growth factors) as active ingredients is well understood in the art. The active therapeutic ingredient is often mixed with excipients or media which are pharmaceutically acceptable and compatible with the active ingredient. In addition, if desired, the composition can contain minor amounts of auxiliary substances such as wetting or emulsifying agents, pH buffering agents which enhance the effectiveness of the active ingredient.

A bioactive agent can be formulated into the composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The compositions are administered in a manner compatible with the dosage formulation, and in a therapeutically effective amount. The quantity to be administered depends, for instance, on the subject and debilitation to be treated, capacity of the subject's organ, cellular and immune system to accommodate the composition, and the nature of the cell or tissue therapy, etc. Precise amounts of composition required to be administered depend on the judgment of the practitioner and are peculiar to each individual. However, suitable dosages of the composition may range from about 0.05-100.0×10⁶ substantially homogeneous GDSC-SP cells/10 mm of treatment site per treatment location per day, preferably about 0.10-50.0×10⁶ substantially homogeneous GDSC-SP cells/10 mm of treatment site per treatment location per day, and more preferably about 0.5-5.0×10⁶ substantially homogeneous GDSC-SP cells/10 mm of treatment site per treatment location per day, and depend on the route of administration and the size of the treatment location. Suitable regimes for initial administration and follow on administration are also variable, but can include an initial administration followed by repeated doses at one or more hour, or day, intervals by a subsequent injection or other administration.

One of skill in the art may readily determine the appropriate concentration of cells for a particular purpose. An exemplary dose is in the range of about 0.05-100.0×10⁶ cells per treatment site per day. In a non-limiting example, approximately 0.5×10⁶ substantially homogeneous GDSC-SP cells/10 mm of treatment site per treatment location per day, are intradermally injected adjacent to, or within, the treatment site.

In other embodiments, the substantially homogeneous population of GDSC-SP cells are administered to the treatment site of a mammal at any time after the appearance of a wound or an injury when tissue regeneration is needed. Precise administration schedules for the therapeutic composition depend on the judgment of the practitioner and the type and extent of the wound or injury and are peculiar to each individual.

The substantially homogeneous population of GDSC-SP, cells differentiated therefrom, disclosed herein can be administered by injection into a target site of a subject, preferably via a delivery device, such as a tube, e.g., catheter. In one embodiment, the tube additionally contains a needle, e.g., a syringe, through which the cells can be introduced into the subject at a desired location. Specific, non-limiting examples of administering cells to subjects may also include administration by subcutaneous injection, intramuscular injection, or intravenous injection. If administration is intravenous, an injectable liquid suspension of cells can be prepared and administered by a continuous drip or as a bolus.

Cells may also be inserted into a delivery device, e.g., a syringe, in different forms. For example, the cells can be suspended in a solution contained in such a delivery device. As used herein, the term “solution” includes a pharmaceutically acceptable carrier or diluent in which the cells remain viable. The use of such carriers and diluents is well known in the art. The solution is preferably sterile and fluid to the extent that easy syringability exists. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions can be prepared by incorporating substantially homogeneous population of GDSC-SP cells or differentiated cells as described herein, in a pharmaceutically acceptable carrier or diluent and, as required, other ingredients enumerated above, followed by filter sterilization.

The cells may be administered systemically (for example intravenously) or locally (for example directly into a myocardial defect under echocardiogram guidance, or by direct application under visualization during surgery). For such injections, the cells may be in an injectible liquid suspension preparation or in a biocompatible medium which is injectible in liquid form and becomes semi-solid at the site of damaged tissue. A conventional intra-cardiac syringe or a controllable endoscopic delivery device can be used so long as the needle lumen or bore is of sufficient diameter (e.g. 30 gauge or larger) that shear forces will not damage the cells being delivered.

Cells may be administered in any manner that permits them to graft to the intended tissue site and reconstitute or regenerate the functionally deficient area.

Support matrices into which the substantially homogeneous GDSC-SP cells can be incorporated or embedded include matrices which are biocompatible, recipient-compatible and which degrade into products which are not harmful to the recipient. These matrices provide support and protection for GDSC-SP cells and differentiated cells in vivo.

Natural and/or synthetic biodegradable matrices are examples of such matrices. Natural biodegradable matrices include plasma clots, e.g., derived from a mammal, collagen, fibronectin, and laminin matrices. Suitable synthetic material for a cell transplantation matrix must be biocompatible to preclude migration and immunological complications, and can be able to support extensive cell growth and differentiated cell function. It must also be restorable, allowing for a completely natural tissue replacement. The matrix can be configurable into a variety of shapes and can have sufficient strength to prevent collapse upon implantation. Recent studies indicate that the biodegradable polyester polymers made of polyglycolic acid fulfill all of these criteria, as described by Vacanti, et al. J. Ped. Surg. 23:3-9 (1988); Cima, et al. Biotechnol. Bioeng. 38:145 (1991); Vacanti, et al. Plast. Reconstr. Surg. 88:753-9 (1991). Other synthetic biodegradable support matrices include synthetic polymers such as polyanhydrides, polyorthoesters, and polylactic acid. Further examples of synthetic polymers and methods of incorporating or embedding cells into these matrices are also known in the art. See e.g., U.S. Pat. Nos. 4,298,002 and 5,308,701.

Attachment of the cells to the polymer may be enhanced by coating the polymers with compounds such as basement membrane components, agar, agarose, gelatin, gum arabic, collagens types I, II, III, IV and V, fibronectin, laminin, glycosaminoglycans, mixtures thereof, and other materials known to those skilled in the art of cell culture. All polymers for use in the matrix must meet the mechanical and biochemical parameters necessary to provide adequate support for the cells with subsequent growth and proliferation.

One of the advantages of a biodegradable polymeric matrix is that angiogenic and other bioactive compounds can be incorporated directly into the support matrix so that they are slowly released as the support matrix degrades in vivo. As the cell-polymer structure is vascularized and the structure degrades, placental stem cells may differentiate according to their inherent characteristics. Factors, including nutrients, growth factors, inducers of differentiation or de-differentiation (i.e., causing differentiated cells to lose characteristics of differentiation and acquire characteristics such as proliferation and more general function), products of secretion, immunomodulators, inhibitors of inflammation, regression factors, bioactive agents which enhance or allow ingrowth of the lymphatic network or nerve fibers, hyaluronic acid, and drugs, which are known to those skilled in the art and commercially available with instructions as to what constitutes an effective amount, from suppliers such as Collaborative Research, Sigma Chemical Co., vascular growth factors such as vascular endothelial growth factor (VEGF), epidermal growth factor (EGF), and heparin binding epidermal growth factor like growth factor (HB-EGF), could be incorporated into the matrix or provided in conjunction with the matrix. Similarly, polymers containing peptides such as the attachment peptide RGD (Arg-Gly-Asp) can be synthesized for use in forming matrices (see e.g U.S. Pat. Nos. 4,988,621, 4,792,525, 5,965,997, 4,879,237 and 4,789,734).

In another example, the cells may be transplanted in a gel matrix (such as Gelfoam from Upjohn Company) which polymerizes to form a substrate in which the stem cells or differentiated cells can grow. A variety of encapsulation technologies have been developed (e.g. Lacy et al., Science 254:1782-84 (1991); Sullivan et al., Science 252:718-712 (1991); WO 91/10470; WO 91/10425; U.S. Pat. No. 5,837,234; U.S. Pat. No. 5,011,472; U.S. Pat. No. 4,892,538). During open surgical procedures, involving direct physical access to the damaged tissue and/or organ, all of the described forms of undifferentiated stem cells or differentiated stem cell delivery preparations are available options. These cells can be repeatedly transplanted at intervals until a desired therapeutic effect is achieved.

In an exemplary embodiment, a therapeutic composition comprising an effective amount of substantially homogeneous population of GDSC-SP cells may be used to treat a subject with a vascular disease. As used herein, “vascular disease” refers to a disease of the human vascular system. Examples include peripheral arterial disease, abdominal aortic aneurysm, carotid disease, and venous disease. The GDSC-SP cells can be used to produce vascular endothelial cells that may be used in methods for remodeling tissue or replacing a scar tissue in a subject. Vascular endothelial cells may also be used to repair vascular damage.

### EXAMPLES

### Example 1

### Isolation of Murine Male Gonadal Stem Cells

The testes of either neonatal mouse pups (2-5 days after birth) or adult mice are sterilely removed from the body. The capsule of the testes is removed and the seminiferous tubules are suspended in an enzyme solution consisting of 1 mg/mL collagenase 1A and 10 units/mL DNase in phosphate-buffered saline (PBS). The testes are digested at 37° C. in a water bath until all tubules are digested. The reaction is stopped with fetal bovine serum (FBS). The supernatant enzyme-FBS solution is removed and cells are resuspended in culture medium and kept on ice until use.

### Example 2

### Isolation of Murine Female Gonadal Stem Cells

Mouse ovaries from 40-60 pups, aged 2-5 days, are dissected under a micro dissection microscope and used for cell isolation. Ovaries are first collected in a culture dish containing cold D-PBS supplemented with 4 mM EDTA. Using a 5 ml pipette, ovaries are then transferred with to a 50 ml conical tube. After centrifugation and washing, the D-PBS wash solution is removed and the ovaries are resuspended in collagenase (1 mg/ml) and DNase-1 (20 unit/ml) and placed in a 37° C. water bath. Every 10 min, the digesting ovarian tissues are physically disrupted by pipette and at the end of the incubation (30 min), 5 ml of FBS is added to neutralize the enzymes. The resultant cell suspension is passed through a 40 μm strainer to remove tissue debris and the isolated cells are collected by centrifugation at 400×G for 10 min. The supernatant enzyme-FBS solution is removed and cells are resuspended in culture medium and kept on ice until use.

### Example 3

### Isolation of Primate Male Gonadal Stem Cells

Human testes collected as testicular biopsies from patients with non-obstructive azospermia or remnant of testes tissue collected after orchiectomy are used for this study. All the tissues are donated with the informed consent of the patients. Tissues are transferred in PBS-antibiotics at 4° C. within 24 hr of collection. The procedure of processing human testicular tissue is similar to that for primate as disclosed below.

Testes from euthanized Rhesus monkeys, age 3-7, are surgically removed; placed in PBS supplemented with penicillin/streptomycin (Cellgro and Invitrogen, respectively) and transported overnight on ice. After surgical removal of the testicular capsule, a biopsy sample is removed for histology and molecular analysis. The remaining seminiferous tubular tissues are finely minced and digested with collagenase A (1 mg/mL) (Roche) and DNase (10 U/mL) (Invitrogen) in a reciprocating 37° C. water bath for 15 min. After collagenase digestion, the undigested tissue is sedimented at unit gravity and cells in the supernatant are removed. The undigested tissue is further digested in an enzyme cocktail consisting of 1.5 mg/mL collagenase A, 1.5 mg/mL hyaluronidase Type V (Sigma), 0.5 mg/mL trypsin (Worthington Biochemical Corporation), and 10 units/mL DNAse in DMEM in a reciprocating 37° C. water bath for 20 min. Digested and undigested tissues are passed through a 70 μm strainer into FBS to inactivate enzymes. After centrifugation at 400×g for 10 min, the cell pellets are resuspended in DMEM+10% FBS and placed in tissue culture coated 15 cm dishes in a 5% CO₂/95% air humidified incubator.

### Example 4

### Identification of Gonadal-Derived Stem Cell Side Population Cells

For GDSC-SP analysis, gonadal cells isolated in Examples 1-3 are suspended in a concentration of 1×10⁶ cells/mL in DMEM with 10% FBS. The cells are incubated with Hoechst 33342 (Sigma) at a final concentration of 2.5 μg/mL. The cells are gently agitated every 20 min in a 37° C. water bath for a total of 90 min. After incubation, cells are pelleted by centrifugation and kept on ice until flow sorting. To demonstrate Hoechst efflux inhibition, cells are incubated with verapamil (Sigma) at a final concentration of 25 μg/mL in addition to Hoechst staining for the same incubation period.

Sorting is done on a Cytopeia InFlux Cell Sorter (Seattle, Wash.). Hoechst-stained cells are excited with a 355 nm 20 mW UV laser (Lightwave Electronics, Mountain View, Calif.) and Hoechst blue and red emission is separated with a 560 nm dichroic mirror and collected using a 460/50 and 670/40 band pass filters, respectively. Fluorescein isothiocyanate (FITC) and phycoerythrin (PE) are excited with a 488 nm 200 mW laser (Coherent, Santa Clara, Calif.) and emission is collected with a 530/40 and 580/30 band pass filters, respectively. Allophycocyanin (APC) is excited with a 638 nm 25 mW laser (Coherent) and emission was collected with a 670/40 band pass filter.

For antibody staining, cells are spun down and concentrated into 500 mL of Hoechst staining buffer and kept on ice. Cells are stained with anti-mouse Sca-1-PE, CD90-APC, CD117-APC, CD34-FITC, and antibodies for lineage determination (BD Biosciences Pharmingen, San Diego, Calif.). The lineage kit contains anti-mouse CD3e, CD11c, CD45R/B220, Ly-76, Ly-6G and Ly-6C all conjugated to APC. Other markers include SSEA4, CD49f and ABCG2/BCRP1. Cells are stained for 30 minutes, washed once in cold Hoechst staining buffer and kept on ice until flow analysis.

The conserved phenomenon of Hoechst 33342 efflux is observed in gonadal tissue. To identify the side population cells, all scatter events are included in the first gate of the scatter plot. Gonadal-derived stem cell SP cells, identified by Hoechst staining, are backgated on the scatter plot. Digested gonadal tissue shows three major populations of cells on the scatter plot. Gonad-derived stem cell SP cells reside in an area related to low side scatter (SSC) and low to mid forward scatter (FSC). Gonad-derived stem cell SP cells have a low SSC, indicating they are smaller than the main population of cells.

The SP phenotype in many tissues is caused by membrane bound protein transporters of the ABC transporter superfamily. To determine whether ABC transporter activity creates the SP phenotype in GDSC-SP cells, verapamil is added to the cell suspension to a final concentration of 25 μg/mL. The addition of verapamil diminishes the efflux of Hoechst dye suggesting that the SP phenotype is due to the ABC transporter.

Gonad-derived stem cell SP cells are stained for surface markers common to many types of side population cells. All markers are direct conjugated antibodies studied with flow cytometry. Negative controls are unstained gonadal cells. Positive staining is defined as fluorescence intensity above 95% of the negative control. Staining not as intense, falling in a range of 30%-80%, is considered low to mid level staining.

Adult stem cells, under tight regulatory control, are mainly quiescent during their life cycle. Freshly sorted gonadal side population cells are stained with propidium iodide to assess their cell cycle status. The data indicates that both the side population and the main population cells are mainly quiescent, with less than 0.5% of the cells in growth phase. This is also true for other cells that stained with Hoechst 33342 at a greater intensity.

Gonad-derived stem cell SP cells are cells that efflux Hoechst 33342 dye and are enriched for stem cells. However, not all GDSC-SP cells express stem cell markers. The GDSC-SP cell population contains cells that have not committed to any lineage.

### Example 5

### Differentiation of GDSC-SP Cells

After sorting, GDSC-SP cells are washed once with DMEM supplemented with 10% FBS and cultured on a feeder layer of mouse embryonic fibroblast STO cells. The STO feeder cells are plated on dishes coated with 0.1% (wt/vol) gelatin, treated with mitomycin C (Sigma) at a concentration of 10 μg/ml for 2.5 h at 37° C., and washed three times with PBS. Sorted GDSC-SP cells are plated onto mitomycin C-treated STO feeder cells with a daily change of culture medium.

In yet another embodiment, GDSC-SP cells were cultured under feeder-free/zenogeneic supplement-free clinical grade culture conditions, using proper matrices for adhesive culture or in a bioreactor in suspension.

To examine the functional capabilities of GDSC-SP cells, they are differentiated into different cell types. For osteogenesis, adipogenesis, and neurogenesis 50,000 cultured GDSC-SP cells at passage four are plated. For chondrogenesis, 80,000 cultured GDSC-SP cells at passage four are plated as a micromass.

Adipogenesis

Fifty thousand cells are grown in adipogenic induction and maintenance medium (Cambrex, Walkersville, Md.) according to manufacturer's specifications. Briefly, cells are plated into a 6 cm dish in DMEM with 10% FBS and allowed to attach. Cells are transferred into adipogenic induction medium for 3 days and changed to adipogenic maintenance medium for 3 days. GDSC-SP cells are cultured in adipogenic medium for three rounds days until fat vacuoles develop. The earliest time in which vacuoles are evident is at 7-10 days. A percentage of the GDSC-SP cells develop fat vacuoles. As the cells become larger, the most obvious morphological change is the appearance of fat vacuoles.

Cells cultured in adipogenic medium are then stained for adipogenic changes. Cells are washed 2× in PBS and fixed in 4% paraformaldehyde overnight at 4° C. Plates are washed three times in 70% ethanol and incubated with oil red 0 staining dye for 5 minutes at room temperature. Plates are washed three times with 70% ethanol and twice with dH₂O to remove excess dye. Hematoxylin is added to visualize cell nuclei for 5 minutes. Plates are washed with dH₂O twice. Staining with oil red 0 shows cells with oil droplets as a deep red color. The control dish has no staining for oil red.

Chondrogenesis

Eighty thousand cells are grown in chondrogenic induction medium (Cambrex) according to manufacturer's specifications. Briefly, cells are pelleted into a micromass in 100 μL of DMEM with 10% FBS and are allowed to attach tightly into a micromass plated in the center of a 6 cm dish. Cells are cultured with chondrogenic induction medium that is changed every two days. While in chondrogenic medium, morphological changes begin as early as 5 days. The cells enlarge and the micromass becomes much more dense. At days 7-8 the micromass condences into a visible pellet and lifts off the culture dish.

At this point the cells are stained with alcian blue reagent to detect proteoglycosylations. Cells are washed 2× in PBS and fixed in 4% paraformaldehyde overnight at 4° C. Cells are incubated with 1% (w/v) alcian blue in 0.1 N HCl. Plates are incubated at room temperature for 1 hour. Plates are washed three times with 0.1 N HCl to remove excess dye. The entire micromass stains deep blue and the surrounding cells also stain blue in their cytoplasm. The control dish has little to no staining.

Osteogenesis

Fifty thousand cells are grown in osteogenic induction medium (Cambrex) according to manufacturer's specifications. Briefly, cells are plated into a 6 cm dish in DMEM with 10% FBS and allowed to attach. Cells are cultured with osteogenic induction medium that is changed every two days. Osteogenis morphological changes begin showing after 10-14 days in culture. Gonadal SP cells become large and cuboidal while undergoing osteogenesis.

After 21 days of culture, osteogenic cells are stained with von Kossa reagent to identify calcified deposits which signify early osteogenesis. Cells are washed 2× in PBS and fixed in 4% paraformaldehyde overnight at 4° C. Plates are washed 2× in dH₂O. Five percent silver nitrate (w/v) is added and the plates are exposed to UV light for 45-60 min. The plates are washed in dH₂O until all the silver nitrate is removed. The plates are counterstained with 2% sodium thiosulfate for 5 minutes. Close to 90% of the cells stain with von Kossa.

Neurogenesis

Fifty thousand cells are grown in DMEM with 20% FBS supplemented with 1 mM β-mercaptoethanol for a total of three days. Medium is changed daily. Morphological signs of neurogenesis are seen as early as two days after culturing. Neuron-like dendritic progections begin to develop and the cell somas begin to undertake pyrimadal morphology, a shape specific for neurons. After 3 days of culture, the cells are stained for nestin. Cells are washed twice in PBS and fixed in 4% paraformaldehyde overnight at 4° C. Plated are blocked for 30 minutes with Fc block (BD Pharmingen) diluted in PBS blocking buffer (1×PBS+10% FBS). Cells are washed three times in PBS-T wash buffer (1×PBS+0.1% Triton X-100) and are further incubated in PBS-T for 30 minutes. Mouse anti-nestin (IgG1) (Chemicon, Temecula, Calif.) is diluted in 1×PBS-T+2% FBS to a final dilution of 1:200 and incubated for 1 hour with constant rotation. Plated are washed twice in PBS-T. Anti-mouse immunoglobulin PE (BD Pharmingen) diluted 1:200 in PBS-T+2% FBS is incubated for 30 minutes for visualization. Approximately 70% of differentiated GDSC-SP cells express nestin.

Cardiogenesis

Fifty thousand cells are grown in DMEM with 10% FBS supplemented with 5-azacytidine (Sigma) to a final concentration of 9 mM. Cells are cultured for 3 days in cardiogenic induction medium then switched to DMEM with 10% FBS and stained for the cardiac marker troponin. Cells are washed 2× in PBS and fixed in 4% paraformaldehyde overnight at 4° C. Plates are blocked for 15 minutes with Fc block (BD Pharmingen) diluted in 1×PBS with 10% FBS. Cells are washed twice in PBS-T wash buffer (1×PBS+0.1% Triton X-100) and are further incubated in PBS-T for 30 minutes. Mouse anti-troponin1 (IgG2a) (Chemicon) is diluted in 1×PBS-T+2% FBS with a final dilution of 1:200 and incubated for 1 hour with constant rotation. Plates are washed twice in PBS-T. Anti-mouse Ig PE (BD Pharmingen) diluted 1:200 in PBS-T+2% FBS is incubated for 30 minutes for visualization.

### Example 6

### Induction of Wound Healing with GDSC-SP Cells in Mice

Approximately 10 cm incisions are made on the back of NOD/Scid mice (3 animals per group) and the wound is left open. At approximately one minute after injury, 50,000 GDSC-SP cells are injected at the site of the injury in one group of mice. A second group of mice receives no cell transplantation (control group). At two weeks after injury, tissue sections of the injury site are taken to evaluate the extent of wound healing and tissue regeneration.

Tissue sections from control mice 14 days after injury demonstrates wound-induced disruption of skin architecture and formation of scar tissue. Tissue sections from mice implanted with GDSC-SP cells demonstrates wound healing of restoration of normal skin architecture without evidence of scar tissue.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter can at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A composition comprising:
a substantially homogeneous population of isolated gonadal-derived stem cell side population cells (GDSC-SP), wherein the substantially homogeneous population of GDSC-SP cells comprises a phenotype in which at least 85% of the cells express all of the cell surface markers SSEA4, ABCG2, CD117, CD34, BCRP1, SCA1, CD90, CD49f, VASA, and GPR-125 and do not express CD45 or lineage markers; and
at least one pharmaceutically acceptable excipient.

2. (canceled)

3. (canceled)

4. (canceled)

5. The composition of claim 1, further comprising at least one bioactive agent.

6. The composition of claim 5, wherein the bioactive agent comprises a growth factor, an anti-rejection agent, an anti-inflammatory agent, an anti-infective agent (e.g., antibiotics and antiviral agents), an analgesic and/or analgesic combination, an anti-asthmatic agent, an anticonvulsant, an antidepressant, an anti-diabetic agent, an anti-neoplastic, an anti-cancer agent, an anti-psychotic, an antioxidant, an immunosuppressive agent, a vitamin, a mineral, or an agent used for cardiovascular diseases such as an anti-restenosis and/or anti-coagulant compound.

7. (canceled)

8. A method of promoting tissue regeneration in a subject in need thereof, the method comprising:
administering a tissue regenerating effective amount of a composition according to claim 1 to a treatment site in the subject thereby inducing tissue regeneration at the treatment site.

9. (canceled)

10. (canceled)

11. (canceled)

12. The method of claim 8, wherein the tissue regenerating effective amount of the substantially homogeneous population of GDSC-SP cells is approximately 0.5×10⁶ cells/10 mm of treatment site per treatment location per day.

13. The method of claim 8, wherein the substantially homogeneous population of GDSC-SP cells are autologous to the subject.

14. The method of claim 8, wherein the substantially homogeneous population of GDSC-SP cells are allogeneic to the subject.

15. The method of claim 8, wherein tissue regeneration is skin regeneration at the site of a wound, cardiac muscle regeneration, neural tissue regeneration, or vascular regeneration.

16. (canceled)

17. The method of claim 8, wherein tissue regeneration minimizes scarring at the site of a wound.

18. The method of claim 8, wherein the administering step comprises at least one method selected from the group comprising of topical application, intradermal injection, intravenous injection, and subcutaneous injection.

19. The method of claim 8, further comprising administering at least one bioactive active agent.

20. The method of claim 19, wherein the bioactive agent comprises a growth factor, an anti-rejection agent, an anti-inflammatory agent, an anti-infective agent (e.g., antibiotics and antiviral agents), an analgesic and/or analgesic combination, an anti-asthmatic agent, an anticonvulsant, an antidepressant, an anti-diabetic agent, an anti-neoplastic, an anti-cancer agent, an anti-psychotic, an antioxidant, an immunosuppressive agent, a vitamin, a mineral, or an agent used for cardiovascular diseases such as an anti-restenosis and/or anti-coagulant compound.

21. (canceled)

22. (canceled)

23. A method of promoting tissue regeneration in a subject in need thereof, the method comprising:
obtaining a substantially homogeneous population of GDSC-SP cells, wherein the substantially homogeneous population of GDSC-SP cells comprises a phenotype in which at least 85% of the cells express all of the cell surface markers SSEA4, ABCG2, CD117, CD34, BCRP1, SCA1, CD90, CD49f, VASA, and GPR-125, and do not express CD45 or lineage markers;
differentiating the substantially homogeneous population of GDSC-SP cells into cells of the same type as the tissue in need of regeneration; and
administering a tissue regenerating effective amount of the substantially homogeneous population of GDSC-SP cells to a treatment site in the subject thereby inducing tissue regeneration at the treatment site.

24. The method of claim 23, wherein administering a tissue regenerating effective amount comprises a composition comprising GDSC-SP cells and at least one pharmaceutically acceptable excipient.

25. (canceled)

26. (canceled)

27. The method of claim 23, wherein the tissue regenerating effective amount of the substantially homogeneous population of GDSC-SP cells is approximately 0.5×10⁶ cells/10 mm of treatment site per treatment location per day.

28. (canceled)

29. (canceled)

30. The method of claim 23, wherein tissue regeneration is skin regeneration at the site of a wound, cardiac muscle regeneration, neural tissue regeneration, or vascular regeneration.

31. (canceled)

32. The method of claim 23, wherein tissue regeneration minimizes scarring at the site of a wound.

33. The method of claim 23, wherein the administering step comprises at least one method selected from the group comprising of topical application, intradermal injection, intravenous injection, and subcutaneous injection.

34. The method of claim 23, further comprising administering at least one bioactive active agent.

35. The method of claim 34, wherein the bioactive agent comprises a growth factor, an anti-rejection agent, an anti-inflammatory agent, an anti-infective agent (e.g., antibiotics and antiviral agents), an analgesic and/or analgesic combination, an anti-asthmatic agent, an anticonvulsant, an antidepressant, an anti-diabetic agent, an anti-neoplastic, an anti-cancer agent, an anti-psychotic, an antioxidant, an immunosuppressive agent, a vitamin, a mineral, or an agent used for cardiovascular diseases such as an anti-restenosis and/or anti-coagulant compound.

36. (canceled)

37. (canceled)

38. The composition of claim 1, wherein the lineage markers comprise CD2, CD3, CD14, CD16, CD19, CD24, CD56, CD66b, and glycophorin A.

38. (canceled)
